(19) United States
(12) Patent Application Publication
(10) Pub. No.: US 2003/0018006 A1
(43) Pub. Date: Jan. 23, 2003

(54) In vivo electroporation-mediated cytokine/immunocytokine-based antitumoral gene

(75) Inventors: Mi-Hua Tao, Shijr City (TW); Shan-Chih Lee, Changhua (TW); Chang-Jer Wu, Taipei (TW)

Correspondence Address: COHEN, PONTANI, LIEBERMAN & PAVANE, 551 Fifth Avenue, Suite 1210, New York, NY, 10176, US

(73) Assignee: Academia Sinica

(21) Appl. No.: 10/188,127

(22) Filed: Jul. 1, 2002

Related U.S. Application Data
(60) Provisional application No. 60/302,422, filed on Jun. 29, 2001.

(51) Int. Cl. A61K 48/00; C12N 15/87

(52) U.S. Cl. 514/44; 435/459

(57) ABSTRACT

The present invention provides a method of treating cancer in a mammal comprising delivering by electroporation an immunocytokine or cytokine gene in an expression plasmid into cells of the mammal.

### RELATED APPLICATIONS

This application claims priority from U.S. Provisional Patent Application Serial No. 60/302,422 which was filed on Jun. 29, 2001.

### BACKGROUND OF THE INVENTION

1. Field of the Invention

The present invention is in the field of treating cancer by delivering by electroporation an immunocytokine or cytokine gene in an expression plasmid into cells of the mammal.

2. Description of the Related Art

Many cytokines, either administered systemically or expressed as transgenes by tumor cells, have been intensively investigated as potential anticancer agents. Among the cytokines evaluated, interleukin-12 (IL-12) has been shown to confer potent antitumor activities. IL-12 is a heterodimeric cytokine that is produced primarily by activated antigen-presenting cells and mediates a broad range of effects on both innate and acquired immunity. Gately M K, Renzetti L M, Magram J, Stern A S, Adorini L, Gubler U, Presky D H. The interleukin-12/interleukin-12-receptor system: role in normal and pathologic immune responses. Annu Rev Immunol 16:495-521;1998. It has been well documented that IL-12 can augment the cytotoxic activities of natural killer (NK) cells and cytotoxic I lymphocytes (CTLs), facilitate type 1 T helper (Th) cell development, and regulate production of many cytokines, particularly for interferon-γ (IFN-γ) production from NK and T cells. Chan S H, Perussia B, Gupta J W, Kobayashi M, Pospisil M, Young H A, Wolf S F, Young D, Clark S C, Trinchieri G. Induction of interferon gamma production by natural killer cell stimulatory factor: characterization of the responder cells and synergy with other inducers. J Exp Med 173:869-79;1991. Germann T, Gately M K, Schoenhaut D S, Lohoff M, Mattner F, Fischer S, Jin S C, Schmitt E, Rude E. Interleukin-12/T cell stimulating factor, a cytokine with multiple effects on T helper type 1 (Th1) but not on Th2 cells. Eur J Immunol 23:1 762-70; 1993. IL-12 also possesses IFN-γ- and IFN inducible protein 10-dependent antiangiogenic activity. Coughlin C M, Salhany K E, Gee M S, LaTemple D C, Kotenko 5, Ma X-J, Gri G, Wysocka M, Kim J E, Liu L, Liao F, Farber J M, Pestka S, Trinchieri G, Lee W M F. Tumor cell responses to IFNγ affect tumorigenicity and response to IL-12 therapy and antiangiogenesis. Immunity 9:25-34;1998. These diverse biological functions make IL-12 a potent therapeutic agent for malignant diseases. Tahara H, Lotze M T. Antitumor effects of interleukin-12 (IL-12): applications for the immunotherapy and gene therapy of cancer. Gene Ther 2:96-106; 1995. Trinchieri G, Scott P. Interleukin-12: basic principles and clinical applications. Curr Top Microbiol Immunol 238:57-78;1999. Administration of recombinant IL-12 locally or systemically has been reported to induce potent antitumor activity in a variety of murine tumor models, causing regression of established tumors. Brunda M J, Luistro L, Warner R R, Wright R B, Hubbard B R, Murphy M, Wolf S F, Gately M K. Antitumor and antimetastatic activity of interleukin 12 against murine tumors. J Exp Med 178:1223-30;1993. Nastala C L, Edington H D, McKinney T G, Tahara H, Nalesnik M A, Brunda M J, Gately M K, Wolf S F, Schreiber R D, Storkus W J. Recombinant IL-12 administration induces tumor regression in association with IFN-gamma production. J Immunol 153:1697-706; 1994. Zou J P, Yamamoto N, Fujii T, Takenaka H, Kobayashi M, Herrmann S H, Wolf S F, Fujiwara H, Hamaoka T Systemic administration of rIL-12 induces complete tumor regression and protective immunity: response is correlated with a striking reversal of suppressed IFN-gamma production by anti-tumor T cells. Intlmmunol 7:1135-45;1995, and inhibiting formation of experimental metastases. Brunda M J, Luistro L, Warner R R, Wright R B, Hubbard B R, Murphy M, Wolf S F, Gately M K. Antitumor and antimetastatic activity of interleukin 12 against murine tumors. J Exp Med 178:1223-30;1993. Nastala C L, Edington H D, McKinney T G, Tahara H, Nalesnik M A, Brunda M J, Gately M K, Wolf S F, Schreiber R D, Storkus W J. Recombinant IL-12 administration induces tumor regression in association with IFN-gamma production. J Immunol 153:1697-706; 1994 and spontaneous metastases Boggio K, Nicoletti G, Carlo E D, Cavallo F, Landuzzi L, Melani C, Giovarelli M, Rossi I, Nanni P, Giovanni C D, Bouchard P, Wolf S, Modesti A, Musiani P, Lollini P L, Colombo M P, Forni G. Interleukin 12-mediated prevention of spontaneous mammary adenocarcinomas in two lines of Her-2/neu transgenic mice. J Exp Med 188:589-596;1998. However, in these studies, repeated delivery of recombinant IL-12 on a daily basis was required to achieve the maximal therapeutic activity, and was also usually associated with a dose-dependent toxicity. Gately M K, Warrier R R, Honasoge S, Carvajal D M, Faherty D A, Connaughton S E, Andersion T D, Sarmiento U, Hubbard B R, Murphy M. Administration of recombinant IL-12 to normal mice enhances cytolytic lymphocyte activity and induces production of IFN-gamma in vivo. Int Immunol 6:157-167;1994. Cohen J. IL-12 deaths: explanation and a puzzle. Science 270:908;1995. Alternatively, recombinant viruses, including retroviruses Zitvogel L, Tahara H, Cai Q, Storkus W J, Muller G, Wolf S F, Gately M, Robbins P D, Lotze M T. Construction and characterization of retroviral vectors expressing biologically active human interleukin-12. Hum Gene Ther 5:1493-506; 1994, pox viruses Meko J B, Yim J H, Tsung K, Norton J A. High cytokine production and effective antitumor activity of a recombinant vaccinia virus encoding murine interleukin 12. Cancer Res 55:4765-70; 1995, and adenoviruses Bramson J L, Hitt M, Addison C L, Muller W J, Gauldie J, Graham F L. Direct intratumoral injection of an adenovirus expressing interleukin-12 induces regression and long-lasting immunity that is associated with highly localized expression of interleukin-12. Hum Gene Ther 7:1995-2002;1996, Chen L, Chen D, Block E, O'Donnell M, Kufe D W, Clinton S K. Eradication of murine bladder carcinoma by intratumor injection of a bicistronic adenoviral vector carrying eDNAs for the IL- 12 heterodimer and its inhibition by the IL-12 p40 subunit homodimer. J Immunol 159:351-9;1997, have been used to deliver IL-12 systemically or by local injection of high-titer virus into the tumor mass. Modification of fibroblasts Tahara H, Zeh H J, 3rd, Storkus W J, Pappo I, Watkins S C, Gubler U, Wolf S F, Robbins P D, Lotze M T. Fibroblasts genetically engineered to secrete interleukin 12 can suppress tumor growth and induce antitumor immunity to a murine melanoma in vivo. Cancer Res 54:182-9;1994. Zitvogel L, Tahara H, Robbins P D, Storkus W J, Clarke M R, Nalesnik M A, Lotze M T. Cancer immunotherapy of established tumors with IL-12. Effective delivery by genetically engineered fibroblasts. J Immunol 155:1393-403;1995, tumor cells Tahara H, Zitvogel L, Storkus W J, Zeh H J, McKinney T G, Schreiber R D. Gubler U, Robbins P D, Lotze M T. Effective eradication of established murine tumors with IL- 12 gene therapy using a polycistronic retroviral vector. J Immunol 154:6466-74; 1995, or dendritic cells Nishioka Y, Hirao M, Robbins P D, Lotze M T, Tahara H. Induction of systemic and therapeutic antitumor immunity using intratumoral injection of dendritic cells genetically modified to express interleukin 12. Cancer Res 59:4035-41; 1999 by viral or nonviral vectors has also been used to deliver IL-12. These alternative approaches for IL-12 delivery have various limitations, such as the induction of host antivector cellular immunity in the adenovirus system, Kozarsky K F, Wilson J M. Gene therapy: adenovirus vectors. Curr Opin Genet Dev 3:499-503; 1993, potential integrational mutagenesis in the retroviral system, Mulligan R C. The basic science of gene therapy. Science 260:926-932;1993, and a relatively low transfection efficiency of nonviral plasmid DNA, even when delivered in complexes with cationic liposomes, Ledley F D. Nonviral gene therapy: the promise of genes as pharmaceutical products. Hum Gene Ther 6:1129-44;1995.

Electroporation (EP) has been widely used to introduce exogenous molecules, including DNA, into cultured cells. Neumann E, Schaefer Ridder M, Wang Y, Hofschneider P H. Gene transfer into mouse lyoma cells by electroporation in high electricfields. EMBO J 1:841-5;1982. Zimmermann U. Electric field-mediated fusion and related electrical phenomena. Biochim Biophys Acta 694:227-77;1982. This system provides much higher transfection efficiency compared with other nonviral transfer systems. EP has also been used for transferring chemotherapeutic agents into tumors in vivo, known as electrochemotherapy. The combination of local injection of an anticancer drug, such as bleomycin, and in vivo EP has been shown to be an effective anticancer treatment in a variety of animal models for different types of cancers Dev S B, Hofmann G A. Electrochemotherapy—a novel method of cancer treatment. Cancer Treat Rev 20:105-15;1994, Nanda G S, Sun F X, Hofmann G A, Hoffman R M, Dev S B. Electroporation enhances therapeutic efficacy of anticancer drugs: treatment of human pancreatic tumor in animal model. Anticancer Res 18:1361-6;1998, Hyacinthe M, Jaroszeski M J, Dang V V, Coppola D, Karl R C, Gilbert R A, Helter R. Electrically enhanced drug delivery for the treatment of soft tissue sarcoma. Cancer 85:409-17; 1999. Moreover, electrochemotherapy for human malignant tumors has achieved significant (33-96%) complete response rates in several clinical trials Hyacinthe M, Jaroszeski M J, Dang V V, Coppola D, Karl R C, Gilbert R A, Helter R. Electrically enhanced drug delivery for the treatment of soft tissue sarcoma. Cancer 85:409-1 7; 1999. Recently, in vivo EP was shown to be effective for introducing reporter genes into a variety of organs and tissues, including mouse muscles Aihara H, Miyazaki J. Gene transfer into muscle by electroporation in vivo. Nat Biotechnol 16:867-70;1998, mouse skin, Titomirov A V, Sukharev 5, Kistanova E. In vivo electroporation and stable transformation of skin cells of newborn mice by plasmid DNA. Biochim Biophys Acta 1088:131-4;1991, mouse myeloma Rots M P, Delteil C, Golzio M, Dumond P, Cros 5, Teissie J. In vivo electrically mediated protein and gene transfer in murine melanoma. Nat Biotechnol 16: 168-71;1998, chicken embryos, Muramatsu T, Mizutani Y, Ohmori Y, Okumura J. Comparison of three nonviral transfection methods for foreign gene expression in early chicken embryos in ovo. Biochem Biophys Res Commun 230:376-80;1997, rat liver, Heller R, Jaroszeski M, Atkin A, Moradpour D, Gilbert R, Wands J, Nicolau C. In vivo gene electroinjection and expression in rat liver. FEBS Lett 389:225-8;1996, rat brain, Nishi T, Yoshizato K, Yamashiro 5, Takeshima H, Sato K, Hamada K, Kitamura I, Yoshimura T, Saya H, Kuratsu J, Ushio Y. High-efficiency in vivo gene transfer using intraarterial plasmid DNA injection following in vivo electroporation. Cancer Res 56:1050-5; 1996, and rat corneal endothelium, Oshima Y, Sakamoto T, Yamanaka I, Nishi T, Ishibashi T, Inomata H. Targeted gene transfer to corneal endothelium in vivo by electric pulse. Gene Ther 5:1347-54;1998. This approach has also been used successfully in animal models for the production of functional proteins, such as erythropoietin, Maruyama H, Sugawa M, Moriguchi Y, Imazeki I, Ishikawa Y, Ataka K, Hasegawa 5, Ito Y, Higuchi N, Kazama J J, Gejyo F, Miyazaki J I. Continuous erythropoietin delivery by muscle-targeted gene transfer using in vivo electroporation. Hum Gene Ther 11 :429-37;2000 and interleukin-5, Aihara H, Miyazaki J Gene transfer into muscle by electroporation in vivo. Nat Biotechnol 16:867-70;1998, from transfected muscle tissues. These studies demonstrate that gene transfer into muscles by in vivo EP is more efficient for production of sustained serum levels of therapeutic proteins than a simple intramuscular DNA injection.

### SUMMARY OF THE INVENTION

This invention is a method for in vivo electroporation (EP)-mediated cytokine/immunocytokine-based gene therapy. EP-mediated in vivo delivery of the murine interleukin-12 (IL-12) gene in an expression plasmid was shown to provide antitumor and antimetastasis activity. EP transfer of the IL-12 plasmid into tibialis anterior muscles with low-voltage and long-pulse (100 V/ 50 msec) currents increased 80-fold more IL-12 production and secretion compared to that induced by a simple intramuscular DNA injection. IL-12 expression was accompanied with a high serum IFN-γ level, indicating an induction of systemic biological effects by EP-mediated IL-12 gene transfer. Using a poorly immunogenic, highly metastatic murine B-cell lymphoma (38C13) as a model, we found that electrotransfer of the IL-12 gene resulted in substantial tumor regression. The antitumor effect was consistently demonstrated in animals with microdiseases as well as in animals bearing large established tumors. Compared with other nonviral gene delivery methods, EP-mediated IL-12 gene therapy exhibited the most significant suppression of 38C13 tumor growth. We also demonstrated that intramuscular electrotransfer of pIL-12 results in complete tumor regression or suppression of subcutaneous tumor growth in another two murine tumor: CT-26 colon adenocarcinoma and B16F1 melanoma. In addition, IL-12 electro gene therapy significantly inhibits systemic metastasis. We also evaluated the therapeutic effect of in vivo EP to deliver anti-idiotype and GM-CSF immunocytokines genes. The specific tumor-targeting ability of immunocytokines is expected to further increase the antitumor therapeutic effect as well as to reduce the systemic toxicity associated with free cytokines. We found that intramuscular electrotransfer of the plasmids encoding anti-idiotype-GM-CSF fusion proteins resulted in better antitumor effect than that achieved by the GM-CSF gene. The therapeutic effect was further increased by removing the immunoglobulin CH2-linked carbohydrates from the immunocytokine to reduce its non-specific binding to Fc receptor-bearing cells. These results provide evidence that intramuscular electrotransfer of the cytokine/immunocytokine genes represent a novel therapeutic strategy for cancer treatment.

The present invention therefore provides a method of treating cancer in a mammal comprising delivering by electroporation an immunocytokine gene in an expression plasmid into cells of the mammal. The cells of the mammal may be muscle or cancer cells. The immunocytokine gene may code for cytokine selected from the group consisting of interleukin-1, interleukin-2, interleukin-3, interleukin-4, interleukin-5, interleukin-6, interleukin-12, interleukin-18, gamma-interferon and GM-CSF. Preferably, the immunocytokine gene codes for a monoclonal antibody specific to an antigen of the cancer cells. The monoclonal antibody may be an IgG, such as S5A8. Preferably, the immunocytokine gene has a point mutation that causes the removal of CH2-linked carbohydrate from the immunocytokine. Additionally, it is preferable that the expression plasmid has a CMV promoter.

The present invention more specifically provides for a method for stimulating an immune response against a tumor in a patient, comprising delivering by electroporation into muscle cells of the patient a plasmid containing a gene coding for S5A8^{N297G}-GM-CSF to produce transformed muscle cells.

The present invention further provides a method of treating cancer in a mammal comprising delivering by electroporation a cytokine gene in an expression plasmid into muscle cells of the mammal. Preferably, the delivery is at a site near an active cancer site.

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

In the drawings:

FIG. 1. Increase in luciferase expression after in vivo EP of mouse muscle. Different doses of pcDNA3/Luc (0, 1, 10 or 100 μg) were injected into the quadriceps muscles (half in each quadriceps muscle) of C3H/HeN mice with EP ( [symbol] ) or without EP (□). Complementary doses of pcDNA3 were administered in such a way that each muscle received a total dose of 50 μg of plasmid DNA. Two days after treatment, the animals were sacrificed and the TA muscles (n=4) were collected to determine the luciferase activity. The luciferase activity was presented as relative light units (R.L.U.) per 25 μg of total muscle lysates. Each value represents the mean R.L.U.±SD. *, P<0.01 and **, P<0.05

FIG. 2. Serum IL-12 levels after pIL-12 transfer with and without in vivo EP.

The bilateral TA muscles of C3H/HeN mice were injected with 100 μg (50 μg of each muscle) of pcDNA3 with EP (○) or pIL-12 plasmid DNA with EP ( [symbol] ) or without EP (Δ). Serum samples were obtained on the indicated days after EP and measured for the presence of IL-12 by a commercial ELISA kit. Each value represents the mean IL-12 concentration±SD from five mice. *, P<0.01 and **, P<0.05 versus pcDNA3 control.

FIG. 3. Effect of pIL-12 dose on muscle IL-12 expression. Different doses of pIL-12 (0, 1, 10 or 100 μg) were injected into the quadriceps muscles (half in each quadriceps muscle) of C3H/HeN mice with EP ( [symbol] ) or without EP (□). Complementary doses of pcDNA3 were administered in such a way that each muscle received a total dose of 50 μg of plasmid DNA. Five days after treatment, the animals were sacrificed and the quadriceps muscles (n=6) were collected to determine the IL-12 expression. Each value represents the mean IL-12 concentration±SD. *, P<0.01 and **, P<0.05.

FIG. 4. Effect of pIL-12 dose on serum IFN-γ production. Mice were treated with different doses [0 μg (○), 1 μg ( [symbol] ), 10 μg (Δ) or 100 μg ( [symbol] )] of pIL-12 as indicated in the legend to FIG. 3 and stimulated with electric pulses. Serum samples were collected on the indicated days after EP and measured for the presence of IFN-γ by a commercial ELISA kit. Each value represents the mean IFN-γ concentration±SD from five mice. *, P<0.01 and **, P <0.05

FIG. 5. Suppression of in vivo growth of 38C13 tumors by i.m. electrotransfer of pIL-12. Syngeneic C3H/HeN mice (n=6) were inoculated s.c. with 1×10³ tumor cells at day 0. Animals were treated with pIL-12 by in vivo EP at doses of 0 μg (○), 1 μg ( [symbol] ), 10 μg (Δ), or 100 μg ( [symbol] ). Complementary doses of pcDNA3 were administered in such a way that all animals received a total dose of 100 μg of plasmid DNA (as in FIG. 3). Tumor growth was measured 3 times a week. The mean tumor volume (A) and the percentage of survivors (B) in each group were determined. SDs (bars) are only given at day 20 for clarity. The data are representative results of two independent experiments.

FIG. 6. Comparison of different pIL-12 transfer methods for inhibition of 38C13 tumor growth. C3H/HeN mice (n=5˜6) were inoculated s.c. with 1×10³ tumor cells at day 0. Three days later, animals were randomly divided into five groups. Control groups included mice that received no treatment (A) or i.m. electrotransfer of 100 μg of pcDNA3 (B). In treatment groups, mice were injected with 100 μg of pIL-12 into the TA muscle with (C) or without (D) in vivo EP. Mice that received gene gun-mediated pIL-12 transfection were also included (E). The tumor volume of the individual mouse was plotted as a function of time after tumor cell inoculation. The data are representative results of three independent experiments.

FIG. 7. Treatment of large established 38C13 tumors by i.m. electrotransfer of pIL-12. C3H/HeN mice (n=5˜6) were inoculated s.c. with 1 x 103 tumor cells at day 0. Mice were treated with 100 μg of pIL-12 by in vivo EP at day 7 (B) or day 14 (C). Animals treated with 100 μg of pcDNA3 at day 7 were included as controls (A). The tumor volumes of individual mice were plotted as a function of time after tumor cell inoculation. The data are representative results of three independent experiments.

FIG. 8. Long-term protection induced by i.m. electrotransfer of pIL-12. C3H/HeN mice that completely eliminated 38C13 tumors by IL-12 electro gene therapy were rechallenged by s.c. injection of 1×10³ 38C13 cells at day 60 after treatment ( [symbol] ). Age matched naïve C3H/HeN mice treated with the same amount of 38C13 cells were included as controls (○). The tumor volume of individual mouse (A) and the percentage of survivors (B) in each group were determined. The data summarize the result of five independent experiments.

FIG. 9. Suppression of the in vivo growth of CT-26 and B16F1 tumors by intramuscular electrotransfer of pIL-12. BALB/c mice (n=7) were inoculated subcutaneously with 1×10⁵ CT-26 cells (A) and C57BL/6 mice (n=5) with 2×10⁵ B16F1 cells (B). Three days later, animals were either untreated or treated by intramuscular injection of 100 μg of pIL-12 or pcDNA3 followed by EP. Tumor growth was measured 3 times a week. The mean tumor volume (A and B, left) and the percentage of survivors (A and B, right) in each group were determined. SDs (bars) are only given at day 30 (A) and 20 (B) for clarity. The data are representative results of two independent experiments.

FIG. 10. Inhibition of lung metastases by intramuscular electrotransfer of pIL-12. Experimental lung metastases were induced by intravenous injection of 2×10⁵ B16F1 cells into C57BL/6 mice (A) or 1×10⁵ CT-26 cells into BALB/c mice (B). Mice were electrotransferred with 100 μg of pcDNA3 or pIL-12 at day 3. Animals were killed at day 21 and the tumor nodules in the lung were counted to measure the metastatic load. Data are presented as mean±SD of 5 mice per group. Photographs of representative lungs from mice in the control group (A and B, left top) and the pIL-12/EP-treated group (A and B, left bottom) are shown. The antimetastatic experiments were repeated two times with the CT-26 tumor model and three times with the B16F1 model, and similar results were obtained in each experiment. *, P<0.01 and **, P<0.05.

FIG. 11. Construction of S5A8-GM-CSF immunocytokines constructs. (A), Schematic representation of the S5A8-GM-CSF genetic constructs. The coding sequence of single-chain anti-Id antibody, S5A8, were ligated upstream of the mouse γ2a constant region gene. In pS5A8-GM and pS5A8 N²⁹⁷G-GM, the genetic fragment encoding the mature peptide of murine GM-CSF was ligated 3′ to the C_{H}3 exon. The point mutation of Asn297 to Gly in pS5A8^{N297G}-GM is indicated. (B) Structure diagram of the single-chain anti-Id-GM-CSF fusion protein.

FIG. 12. Immunoblot analysis of the various S5A8-GM-CSF immunocytokines. Balb/3T3 cells were transiently transfected with p3224-3 (lane 1 and lane 4), pS5A8-GM (lane 2 and lane 5), pS5A8^{N297G}-GM (lane 3 and lane 6), or pS5A8 (lane 4 and lane 7). Twenty-four hours after transfection, proteins were concentrated from supernatant with protein A sepharose. The precipitates under reducing (A) or nonreducing (B) conditions were subjected to SDS-PAGE followed by electroblotting to nitrocellulose. Nitrocellulose strips were reacted with goat antimouse IgGγ2a (A) or rat antimouse GM-CSF antibody (B) and detected with horseradish peroxidase-conjugated second-step reagents.

FIG. 13. In vitro production and functional activity of the S5A8-GM-CSF immunocytokines genes. (A), ELISA analysis of proteins produced by S5A8-GM-CSF immunocytokines genes. Microtiter plates were coated with purified 38C13 idiotypic protein. Serial dilution of culture supernatants from Balb/3T3 cells transfected with p3224-3, pS5A8-GM or pS5A8^{N297G}-GM was added to each well and incubated overnight at 4° C. The bound proteins were detected by biotinylated anti-mouse GM-CSF Abs. (B), GM-CSF bioactivity of the S5A8-GM-CSF immunocytokines. NFS-60 cells were incubated with serial dilution of the culture supernatants of the transfected Balb/3T3 cells. Cell proliferation was measurement by ³H-thymidine uptake 16 to 24 hours later. All results are expressed as the mean cpm incorporated ±SD of triplicate.

FIG. 14. Muscle GM-CSF levels after in vivo EP delivery of S5A8-GM-CSF immunocytokines genes. The bilateral TA muscles of C3H/HeN mice were injected with 50 μg of p3224-3 (Δ), pGM-CSF ( [symbol] ), pS5A8-GM (□) or pS5A8^{N297G}-GM ( [symbol] ) with EP. Muscle samples were obtained on the indicated days after EP and measured for the presence of GM-CSF by a commercial ELISA kit. Each value represents the mean GM-CSF concentration±SD from five mice. *, P<0.01 and **, P<0.05 versus p3224-3 control.

FIG. 15. Suppression of in vivo growth of 38C13 tumors by i.m. electrotransfer of S5A8-GM-CSF immunocytokine genes. Syngeneic C3H/HeN mice (n=10) were inoculated s.c. with 1×10³ tumor cells at day 0. One day later, animals were randomly divided into four groups and injected with 100 μg of p3224-3, pS5A8-GM, pS5A8^{N297G}-GM, or pGM-CSF into the TA muscle with in vivo EP. Tumor growth was measured 3 times a week. The percentage of survivor was calculated.

### DETAILED DESCRIPTION OF THE PRESENTLY PREFERRED EMBODIMENTS

Materials and Methods

Mice

Female C3H/HeN, BALB/c, and C57BL/6 mice, 10 weeks old, were purchased from National Laboratory Animal Breeding and Research Center (Taipei, Taiwan) and housed at the Laboratory Animal Facility, Institute of Biomedical Sciences, Academia Sinica (Taipei, Taiwan). All animal studies were approved by the Animal Committee of the Institute of Biomedical Sciences, Academia Sinica and were performed according to their guidelines.

Cell Lines

38C13 murine B-cell lymphoma is a carcinogen (DMBA)-induced tumor originally produced in a T-cell-depleted C3H/eB mouse (1). CT-26, a murine colon adenocarcinoma cell line derived from BALB/c mice treated with N-nitroso-N-methylurethane (2), and B16F1, a malignant melanoma cell line (3), syngeneic in C57BL/6 mice, were obtained from the American Type Culture Collection (Rockville, Md.). Cell lines were maintained in RPMI 1640, 10% heat-inactivated fetal calf serum (FCS), 2 mmol/L L-glutamine, 100 U/mL penicillin, and 100 μg/mL streptomycin (all from Sigma Chemical Co., St. Louis, Mo.) at 37° C., 5% CO₂ in a humidified incubator. 38C13 cells were grown in the above-noted medium supplemented with 50 μmol/L 2-ME.

Plasmids and DNA Preparation

The expression vector, pcDNA3/Luc, pIL-2, pIL-4, pIFN-γ, pGM-CSF, pIL-18, pIL-12, and pTCA3 were previously constructed in our laboratory. The procedures for its construction and expression have been previously described (4, 5). Plasmid DNA was purified from transformed Erichia Coli strain DH5α by Qiagen Plasmid Giga Kits (Qiagen, Hilden, Germany) according to the manufacturer's instructions and stored at −70° C. as pellets. The DNA was reconstituted in sterile saline at a concentration of 1 mg/ml for experimenal use.

Construction of Immunocytokine Expression Vectors

The single chain Fv gene of anti-Id mAb (S5A8) in V_{L}-V_{H} orientation were obtained from pUC9-scFvS5A8 (6) by PCR amplification using upstream primer containing an EcoRI site overlapping the translation start codon ATG and downstream primers containing SfiI site located in the V_{H} region. The EcoRI-SfiI fragments were gel-purified and replaced the B7.2 gene in the yeast expression vector pPICZαA-B7.2-Fc-GMCSF (kindly provided by Chuan-Cheng Wang, M.D.), which contains the mouse γ2a constant region (C γ2a) followed by two Gly codons and the mature murine GM-CSF sequence. Finally, the scFvS5A8-mouse γ2a-GMCSF fusion gene was digested with EcoRI and NotI and then cloned into the restriction enzyme cassette of p3224-3 to create a pS5A8-GM mammalian expression vector. To make an aglycosyl immunocytokine expression vector pS5A8^{N297G}-GM, plasmid pPICZαA-B7.2-Fc^{N297G}-GMCSF (kindly provided by Chuan-Cheng Wang, M.D.), which contains the mutant mouse C γ2a gene with Asn²⁹⁷ replaced with Gly and the GM-CSF sequence, was applied for the cloning according to the procedures described above. To construct the single-chain S5A8 anti-Id antibody, a synthetic oligo primer pairs containing a stop codon was used to replace the GM-CSF sequence in plasmid pS5A8-GM to produce plasmid pS5A8.

SDS-PAGE and Immunoblot Analysis

pS5A8, pS5A8-GM, and pS5A8^{N297G}-GM were transiently transfected into Balb/3T3 cells with Lipofectamine2000 (GIBCO BRL, Gaithersburg, Md., USA). Twenty-four hours after transfection, 1 ml culture supernatants were mixed with 100 μl protein A-Sepharose CL-4B (20% v/v) at 4° C. for 1 hour. The immunoprecipitates were collected by centrifugation, washed three times with 0.5 M NaCl and resuspended in 200 μl SDS-sample buffer with or without 15% 2-mercaptoethanol. These proteins were then subjected to 12% SDS-PAGE, and electrotransferred to nitrocellulose membrane. Blots were probed with either biotinylated 38C13 Id at 1 μg/ml or biotinylated goat antimouse IgGγ2a or biotinylated rat antimouse GM-CSF antibody at 1:500 (PharMingen, San Diego, Calif.) and then reacted with horseradish peroxidase-conjugated streptavidin (PharMingen). All immunoblots were developed with the ECL system (Ametsham, Little Chalfont, UK). The luminescent light emission was recorded on X-ray film.

Intramuscular DNA Injection and Electroporation

Mice were anesthetized with acepriomazine meleate (TechAmerica TM). Fifty microgram of plasmid DNA were injected into the bilateral tibialis anterior (TA) muscles using a disposable insulin syringe with a 27-gauge needle. A total of 100 μg of plasmid DNA was injected in to each mice. Immediately after the injection, a pair of electrode needles was inserted into the muscle to a depth of 5 mm to encompass the DNA injection sites, and electric pulses were delivered using an electric pulse generator (Electro Square Porator ECM830; BTX, San Diego, DA). The shape of the pulse was a square wave. Electrodes consisted of a pair of gold-plated stainless steel needles of 5 mm in length and 0.8 mm in diameter, fixed with a distance between them of 5 mm. Six pulses of 100 volt each were administered to each injection site at a rate of one pulse per sec, with each pulse being 50 msec in duration.

Gene Gun-Mediated In Vivo Gene Transfer

The experiments utilized a hand-held, helium-driven Helios gene delivery system (Bio-rad, Hercules, Calif.). Plasmid DNA was precipitated onto 1.6 μm average diameter gold particles. Particles were suspended in a solution of 0.1 mg of polyvinyl pyrrolidone per ml in absolute ethanol. This DNA/gold/particle preparation was coated onto the inner surface of a Tefzel tubing by using a tube loader (Bio-rad), and the tubing was cut into 0.5-inch segments to result in delivery of 0.5 mg gold and 1.25 μg plasmid DNA per transfection. For tumor therapy, mouse skin overlying and surrounding the target tumor was transfected in vivo with pcDNA3 or pIL-12 starting from day 3 after subcutaneously (s.c.) implantation of tumor cells. Each treatment consisted of four transfections (5 μg plasmid DNA/treatment) with a 300 psi helium gas pulse. One transfection was directly over the tumor site, and three additional treatments were evenly spaced around the circumference of the tumor in a triangle pattern.

Collection and Processing of Tissues

At various times after DNA transfer, blood samples were collected from the tail vein of mice; or, mice were sacrificed and the entire TA muscle was collected for the preparation of a tissue extract. The muscles were immersed in the liquid nitrogen bath then ground into powder using the mortar and pestle. The muscle powder were collected in phosphate buffer saline (PBS, lml per muscle) containing Complete™, a proteinase inhibitor cocktail (Boehringer Mannheim, Germany), and sonicated before collecting the supernatant. Protein concentrations of the muscle extracts were determined by a bicinchoninic acid-based protein assay (Pierce, Rockford, Ill.) and normalized to 3 mg/ml. Aliquots were stored at −20° C. until analyzed.

Luciferase Assay

Animals were euthanized and the entire TA muscle was collected from each mouse leg and immediately frozen in 1.5 ml eppendorf tubes. Tissue samples were stored at −80° C. until processing. The frozen muscles were immersed in the liquid nitrogen bath then ground into powder using the mortar and pestle. Harvest the muscle powder to a micrcentrifuge tube and add 200 μl 1X Reporter Lysis Buffer (Promega, USA). Rock and incubate the tube at room temperature for 15 minutes. The muscle lysates were briefly centrifuged to pellet large debris and the protein concentrations of the supernatants were measured by the BCA protein assay reagent (Pierce, USA). The 20 μl cell lysates (25 μg total protein) were reacted with 100 μl of luciferase assay reagent (Promega, USA) at room temperature in a luminometer. The activity of plasmid expression was quantitated as a relative luciferase activity unit (RLU) per 25 μg of the total protein extract.

ELISA

Cytokine levels in muscle extracts and serum were measured by sandwich ELISA kits purchased respectively from R&D systems (mIL-12 p70 DuoSet ELISA kit; Minneapolis, Minn.) and PharMingen (GM-CSF and IFN-γ ELISA kit, San Diego, Calif.), according to the supplier's instructions.

The Id-binding ability of the various S5A8 immunocytokines was determined by an enzyme-linked immunosorbent assay (ELISA). Microtiter plates were coated with 1 μg/ml of purified 38C13 Id and blocked with 10% bovine calf serum in PBS (BCS/PBS). Serial dilution of supernatants from plasmid-transfected Balb/3T3 cells was added to each well and incubated overnight at 4° C. The final volume was 100 μl in each well. The bound proteins were detected by biotinylated anti-mouse GM-CSF Abs and then detected with alkaline phosphatase-conjugated streptavidin (PharMingen), developed with p-nitrophenyl phosphate (Sigma, St Louis, Mo.) as the substrate, and absorbance at 405 nm was measured using an ELISA plate reader.

Cytokine Proliferation Assays

The biological activity of S5A8-GM-CSF immunocytokines was assayed by their ability to support the proliferation of a murine GM-CSF-dependent NSF-60 cells (7). NSF-60 cells were washed three times with PBS and plated at 5×10³ cells per well in 0.1 ml RPMI-1640 containing 10% heat inactivated fetal bovine serum. Serial dilutions of the test samples were then added to each well. Cells were incubated for 18 hours, then 1 μCi [³H]-thymidine (Amersham) was added to each well and incubation continued for an additional 6 hours. Cells were then collected and the amount of radioactivity determined in a liquid scintillation counter. Recombinant murine GM-CSF (PharMingen) was included in the assay as a positive control.

Murine Tumor Model

Exponentially growing tumor cells were harvested and used for induction of s.c. tumor or metastasis only if their viability exceeded 95%, as determined by trypan blue staining. To generate s.c. tumors, mice were injected s.c. with 1×10³ 38C13 tumor cells (or 1×10⁵, in the case of B16F1 or CT-26 tumor) in 100 μl PBS. Tumor growth was measured every two to three times a week, and the tumor size (in cubic millimeters) was approximated by using the ellipsoidal formula: length (mm)×width (mm)×height (mm)×0.52 (derived from π/6) (8). The mean volume and SD of each group were calculated. Animals were observed until the s.c. tumors measured more than 3,000 mm³ or until any mouse was observed to be suffering or appeared to be moribund. Animals under these conditions were euthanized humanely, according to institutional policy. Sacrifice dates were recorded, and the mean survival of each group was calculated. To generate lung metastasis, 2×10⁵ B16F1 or CT-26 tumor cells were injected i.v. into C57BL/6 or Balb/c mice, respectively. Three weeks after injection, mice were sacrificed and lung metastasis were evaluated by counting tumor nodules on the lung surface. All metastasis counts were performed on dissected lung lobes under a stereoscopic microscope. The statistical significance of differential findings between experimental groups of animals was determined by the Student's t test. Findings were regarded as significant if two-tailed P values were≦0.05.

RESULTS

Part I. Inhibition of Established Subcutaneous And Metastatic Murine Tumors by Intramuscular Electroporation of the Interleukin 12 Gene

Enhancement of Luciferase Gene Expression In Vivo by Electroporation

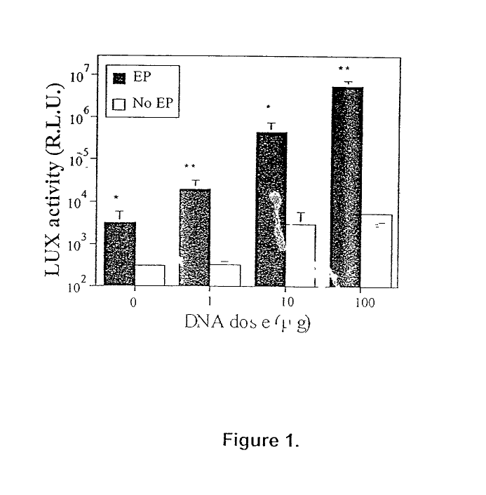

Previous reports have demonstrated that application of electroporation after injection of plasmid DNA has resulted in increased expression of the encoded proteins in the injected muscles (9, 10). To confirm this result, we measured the in vivo expression level of the plasmid pcDNA3/Luc that encodes the reporter protein luciferase. Syngeneic mice C3H/HeN were given a single injection in the TA muscles of various doses (50, 5, 0.5, or 0 μg) of pcDNA3/Luc with or without EP, and the luciferase activities in the injected muscles were assayed 2 days after injection. As shown in FIG. 1, electroporation increased luciferase gene expression at all doses of DNA. For example, at the dose of 50 μg DNA, electrostimulation increased the transgene expression by approximately 1000-fold from a level of 5,429±311 R.L.U. by a simple i.m. injection to 5,408,613±821,160 R.L.U. We also observed that the transgene expression increased as a function of the amount of DNA injected. These data confirm that in vivo EP effectively enhanced the efficiency of muscle-targeted luciferase gene expression in vivo.

IL-12 Expression by i.m. Electrotransfer of pIL-12

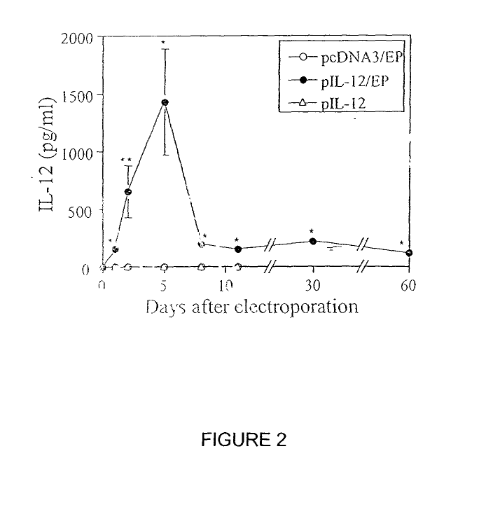

We previously made a bicistronic plasmid, pIL-12, containing the p35 and p40 coding sequences of murine IL-12. I.m. Injection of pIL-12 produces biologically active IL-12 and helps promote cellular immunity to a hepatitis B virus DNA (5). To evaluate the effect of pIL-12 as a potential cancer gene therapy, we delivered the IL-12 gene by in vivo EP, which has been shown to dramatically increase gene expression in muscle tissue (11). C3H/HeN mice were injected in the TA muscles with 100 μg of pIL-12, and one group of mice was electrostimulated immediately after injection. Mice that received the control plasmid pcDNA3 followed by electrostimulation served as negative controls. The time course of gene expression was determined by following serum IL-12 levels. As shown in FIG. 2, no serum IL-12 was detectable within the sensitivity limit of our ELISA assay (<10 pg/ml) in mice that received the control plasmid. Mice in the IL-12 gene-treated but unstimulated group also did not produce detectable serum IL-12. In contrast, the serum IL-12 level in mice treated with the IL-12 plasmid with EP increased from 150±30 pg/ml on day 1 to the peak level 1430±460 pg/ml on day 5 and subsequently decreased to 150±40 pg/ml on day 11. This low but significant level of serum IL-12 persisted for at least 60 days after a single electrotransfer of the IL-12 plasmid (FIG. 2).

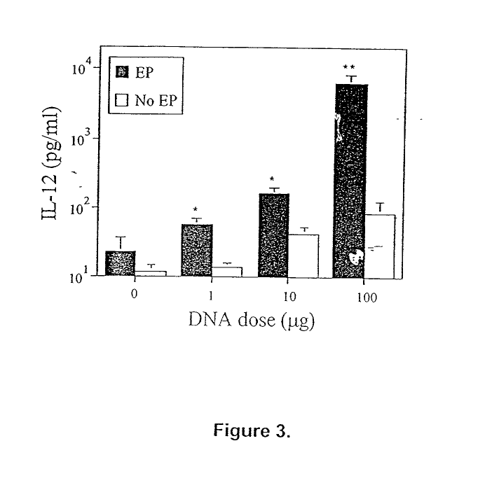

We also analyzed IL-12 expression in muscle tissue following IL-12 gene treatment. The animals were given an injection of 100, 10 or 1 μg of pIL-12 (half in each quadriceps muscle) with or without EP, and the IL-12 levels in selected muscles were assayed at day 5 postinjection. FIG. 3 shows that a low but significant level of IL-12 expression was present in animals treated with 10 or 100 μg of pIL-12 without electrostimulation. Electroporation of the DNA-treated muscle increased the transgene expression at all doses of pIL-12 tested. At the dose of 100 μg, a simple i.m. injection of pIL-12 produced 80±40 pg/ml of IL-12. This level was increased approximately 80-fold to 6340±1980 pg/ml after EP.

Taken together, these data indicate that in vivo EP effectively enhanced the efficiency of muscle-targeted IL-12 gene transfer and, moreover, that continuous delivery of low but sustained level of IL-12 can be achieved by a single plasmid injection using the EP method.

Induction of Serum IFN-γ by i.m. Electrotransfer of pIL-12

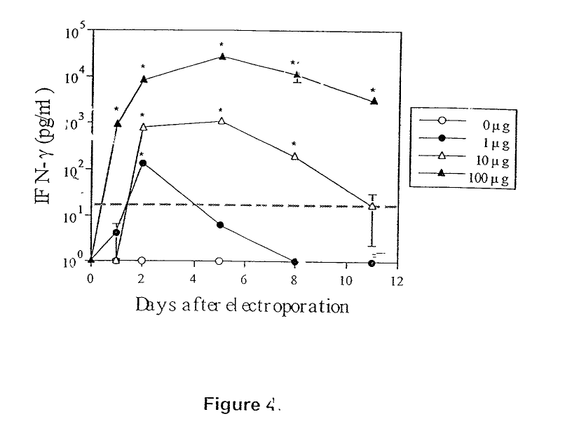

One of the most important properties of IL-12 is its ability to induce the production of IFN-γ from resting and activated T and NK cells. This activity of IL-12 is central to many of the effects seen when IL-12 is administered in vivo, and provides a mechanism whereby IL-12 plays an important role in innate, as well as adaptive, immunity (12). To evaluate whether electrotransfer of pIL-12 could express functionally active IL-12 and enhance IFN-γ production in vivo, serum IFN-γ levels of mice receiving various amounts of pIL-12 were measured over time. Mice treated with pIL-12 without EP did not produce detectable serum IFN-γ levels even at the largest dose (100 μg) of DNA tested (data not shown). In contrast, EP stimulated serum IFN-γ production at all doses of DNA. The circulating IFN-γ reached a peak level at 2 to 5 days after the IL-12 gene treatment, and decreased variably in the different groups from 1/10 to 1/100 of the peak value after 11 days (FIG. 4). The serum IFN-γ measured at different time points correlated well with the amount of DNA injected. At day 5 after EP, pIL-12 at doses of 100 and 10 μg produced 28170±10870 and 1110±570 pg/ml of serum IFN-γ, respectively. A lower dose of the plasmid (1 μg of DNA) only produced a low titer (130±50 pg/ml) of IFN-γ at day 2, with the titer decreasing to an undetectable level at day 5. Mice that received an injection of 100 μg of the control pcDNA3 plasmid with EP did not produce detectable serum IFN-γ (data not shown). This result demonstrates that EP-mediated IL-12 gene transfer was able to produce a substantial quantity of serum IFN-γ, and thus may be capable of systemically stimulating immune cells.

EP-mediated Transfer of the IL-12 Gene Inhibits Tumor Growth

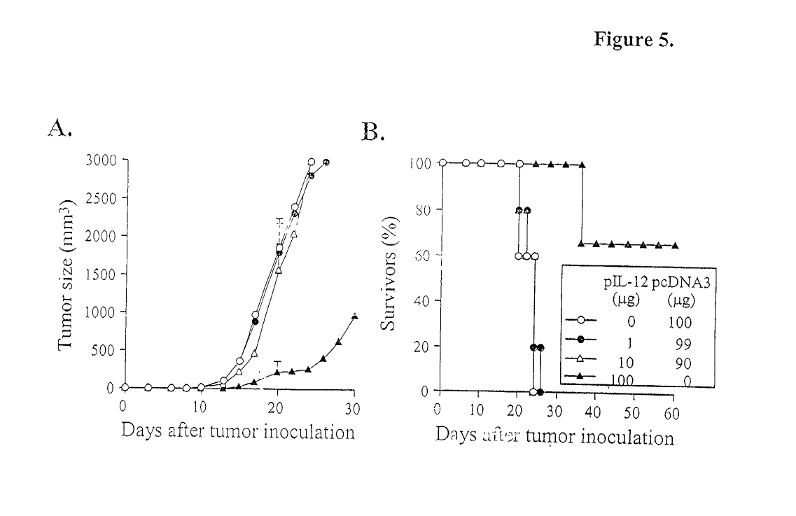

The antitumor effect of IL-12 gene electrotransfer was next evaluated. Syngeneic C3H/HeN mice were inoculated s.c. with 1×10³ 38C13 B cell-lymphoma cells at day 0. Three days later, pIL-12 at doses of 1 μg, 10 μg, or 100 μg was injected into the TA muscles followed immediately by in vivo EP. Complementary doses of pcDNA3 were administered in such a way that all groups of animals received a total dose of 100 μg of plasmid DNA. Mice treated with 100 μg of pcDNA3 alone were included as controls. The tumor volume progression curves and the percentage of survivors are shown in FIG. 5A and FIG. 5B, respectively. Compared with the control group, i.m. electrotransfer of 100 μg of pIL-12 resulted in 66% (4 of 6 mice) long-term survivors (>60 days, P<0.001), whereas all animals in the control group had detectable tumors by day 13. In addition, objective tumor growth suppression was observed in tumor-bearing animals in the pIL-12 (100 μg) group (mean survival time 52±5 days versus 22±1 days for the pcDNA3 control group, P<0.001) (FIG. 5A). By day 20, the mean tumor volume of the two tumor-bearing animals was 223±216 mm³ as compared with 1,863±491 mm³ in the pcDNA3 control group. The long-term survivors remained disease free for an observation period of 120 days and were free of residual or dormant tumor cells as determined by FACS analysis and in vitro culture of spleen and lymph node cells (data not shown). There was no clear dose-effect relationship in the EP-mediated IL-12 gene therapy. Treatment with 1 μg or 10 μg of pIL-12 did not significantly inhibit tumor growth with mean survival time of 23±1 days and 24±1 days, respectively, and produced no long-term survivors (FIGS. 5A and B). Therefore, 100 μg of pIL-12 were used for electroporation in subsequent experiments.

Comparison of Different Transfer Methods for Their Ability to Inhibit Tumor Growth

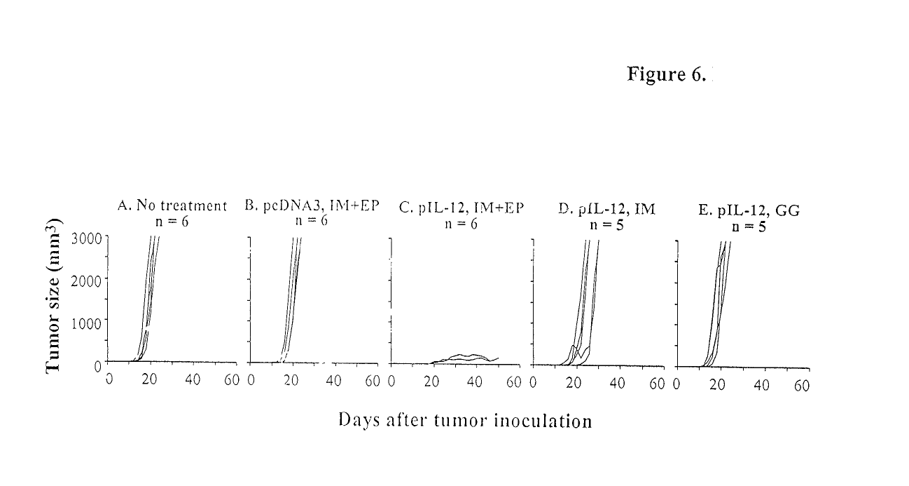

We next compared three different nonviral techniques, i.e. direct muscle injection of plasmid DNA, particle-mediated (gene gun) gene transfer and in vivo EP, for their antitumor activity. Three days after 38C13 tumor inoculation, animals were randomly divided into five groups. Mice were injected with 100 μg of pIL-12 into the TA muscles with or without EP or transfected with 5 μg of pIL-12 by gene gun on the skin overlaying and surrounding the target tumor. Mice receiving no treatment or i.m. electrotransfer with 100 μg of plasmid pcDNA3 were included as controls. As shown in FIG. 6, treatment with the control pcDNA3 plasmid by in vivo EP did not show any inhibition of 38C13 tumor growth (mean survival time 23±2 days versus 22±1 days of the no treatment group, P>0.05). Mice that received simple i.m. injection of pIL-12 (FIG. 6D) led to tumor suppression to some extent, resulting in an additional 5 days of mean survival time (27±3 days), which was not statistically different from the pcDNA3 control (P>0.05). In contrast, pIL-12 delivered by in vivo EP (FIG. 6C) produced 66% (4 of 6 mice) long-term survivors, and resulted in objective tumor growth suppression in the two tumor-bearing animals that eventually died due to systemic tumor metastasis. Interestingly, the IL-12 gene delivered by gene gun at day 3 after tumor inoculation did not produce any therapeutic antitumor effect (FIG. 6E, mean survival time 22±1 days). This result was in contrast to a previous report that demonstrated a significant antitumor effect by gene gun delivery of the IL-12 gene (13). To further confirm this result, tumor-bearing mice were treated by daily gene gun transfection of 5 μg of pIL-12 over a period of 5 days. Compared with the control animals, gene gun-mediated multiple transfection of the IL-12 gene led to minor tumor suppression (mean survival time 29±8 days, P>0.05), but resulted in only 20% (1 of 5 mice) long-term survivors. Taken together, EP-mediated IL-12 gene therapy exhibited the most significant suppression of 38C13 tumor growth as compared with other nonviral gene delivery methods.

Suppression of Established Tumor Growth by i.m. Electrotransfer of pIL-12

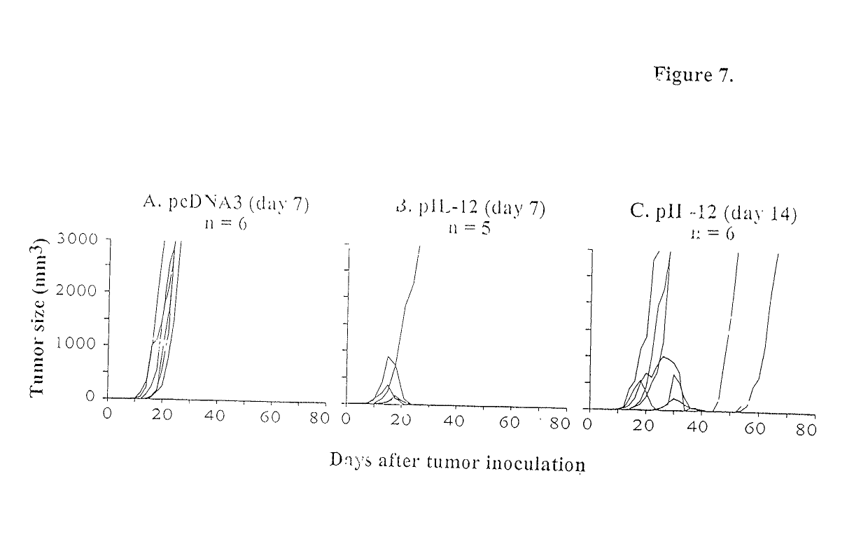

To further evaluate the antitumor effects of pIL-12 electrotransfer, we performed a more stringent experiment on animals bearing advanced 38C13 tumors. In untreated mice, 38C13 tumor grew rapidly after s.c. injection of 1000 tumor cells, reached 2.5 to 3 cm in diameter in 15 to 20 days, and the injected mice died around day 22 to 25. Systemic tumor cell involvement of lymphoid tissues (lymph nodes, spleen and thymus) and nonlymphoid organs (liver and kidney) developed at two weeks after tumor inoculation (data now shown). To test the therapeutic effect of pIL-12 electrotransfer on large established tumors, C3H/HeN mice were s.c. injected with 1000 38C13 tumor cells and EP transfer of the IL-12 gene was started at day 7 or 14 after tumor cell inoculation. Mice electrotransfer of pcDNA3 at day 7 after tumor cell inoculation were served as controls. Compared with the pcDNA3 control group, objective tumor growth suppression was observed in both the day 7- and day 14-treated groups (FIG. 7). In the day 7-treated group, complete tumor regression was observed in most animals, and 80% (4 of 5 mice) of them survived and remained tumor free for more than 80 days (mean survival time 70±23 days versus 25±1 days for the pcDNA3-treated group, P<0.01). Significant tumor regression was also observed in the day-14 treated group, at which time all the animals bore large tumors (approximately 1000 cm³) and had metastases in many organs (up to 12% of the total lymph node cells and 3% of the spleen cells), and usually succumbing to death within a week. A single treatment of pIL-12 by EP at day 14 resulted in regression of 50% (3 of 6 mice) of the large s.c. tumors and significantly prolonged the life-span of these animals (mean survival time 42±18 days, P<0.05) (FIG. 7C). However, there were no long-term survivors in this group; all animals died by day 80. These results demonstrate that i.m. electrotransfer of the IL-12 gene also produced marked therapeutic effects on large established tumors.

Long-term Antitumor Immunity Induced by i.m. Electrotransfer of pIL-12

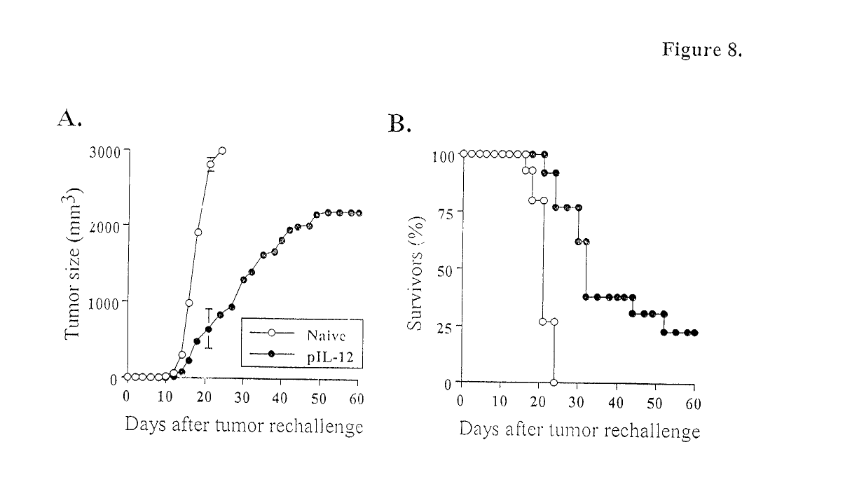

At day 60, we rechallenged animals in which 38C13 tumors were eliminated after treatment with pIL-12 by in vivo EP. Age-matched naïve C3H/HeN mice that received a s.c. injection of 1×10³ 38C13 tumor cells on the same day served as controls. The results of five independent experiments is shown in FIG. 8. Significant suppression of tumor growth was observed in the pIL-12-cured group (mean survival time 40±4 days versus 21±1 days for the naïve group, P<0.0001) (FIG. 8A). Moreover, 25% (4 of 16 mice) of the pIL-12-cured mice survived and remained tumor free for an additional 60 days (FIG. 8B), whereas all animals (15 of 15 mice) in the naïve group succumbed to death by day 24 after tumor cell challenge. These data demonstrate that a single i.m. electrotransfer of pIL-12 can induce long-term protection in this low-immunogenic tumor model.

Suppression of Colon Cancers and Melanoma by Intramuscular Electrotransfer of pIL-12

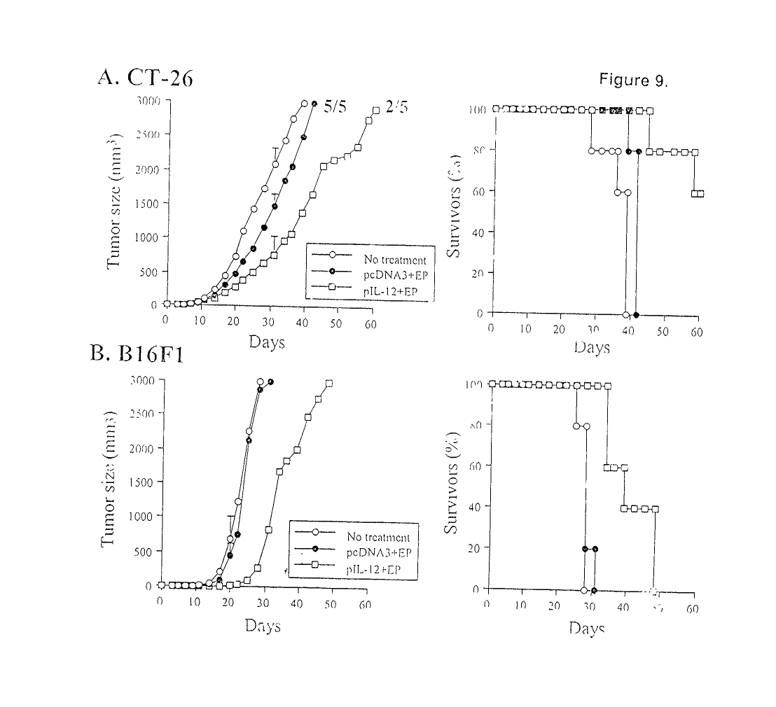

To further evaluate the antitumor effect of IL-12 electro gene therapy, experiments were extended to two additional murine models: CT-26 colon adenocarcinoma and B16F1 melanoma. We first analyzed the therapeutic effect of EP-delivered pIL-12 on subcutaneously injected tumors. Groups of BALB/c and C57BL/6 mice were subcutaneously inoculated with 1×10⁵ CT-26 or 2×10⁵ B16Fl cells, respectively, and 100 μg of pIL-12 or pcDNA3 plasmid was delivered into the quadriceps muscles with EP three days following tumor inoculation. Untreated mice served as controls. As summarized in FIG. 9, a single intramuscular electrotransfer of 100 μg of pIL-12 resulted in complete tumor regression or suppression of tumor growth in these two tumor models. In mice bearing CT-26 tumors, complete tumor regression was achieved in 57% of the tested animals with 4 of 7 mice surviving and remaining tumor free for more than 70 days. In addition, objective tumor growth suppression was observed in tumor-bearing animals in the pIL-12/EP group (mean survival time 63±4 days versus 41±1 days for the pcDNA3 control group, P<0.001) (FIG. 9A). By day 30, the mean tumor volume of the two tumor-bearing animals was 760±315 mm³ as compared with 1,487±180 mm³ in the pcDNA3 control group. In contrast, tumors grew progressively in all the untreated mice or mice received i.m. electrotransfer of pcDNA3 control plasmid, all animals died by day 40. In mice bearing B16F1 melanoma, significant suppression of tumor growth was also observed in the pIL-12/EP-treated group (mean survival time 40±3 days versus 27±1 days for the pcDNA3 control group, P<0.001) (FIG. 9B). However, the suppression of B16F1 tumor growth was transient and all animals eventually died by day 50 from progressing tumors. On day 20 post-B16F1 tumor cell inoculation, the mean tumor volume in mice treated with pIL-12 was 12±12 mm³ versus 455±150 mm³ in mice treated with pcDNA3 (p<0.01). However, the suppression of B16F1 tumor growth was transient and all animals eventually died by day 50 from progressing tumors. Taken together, these results demonstrate that i.m. electrotransfer of the IL-12 gene has marked therapeutic effect on several murine tumor models.

Suppression of Tumor Metastasis by i.m. Electrotransfer of pIL-12

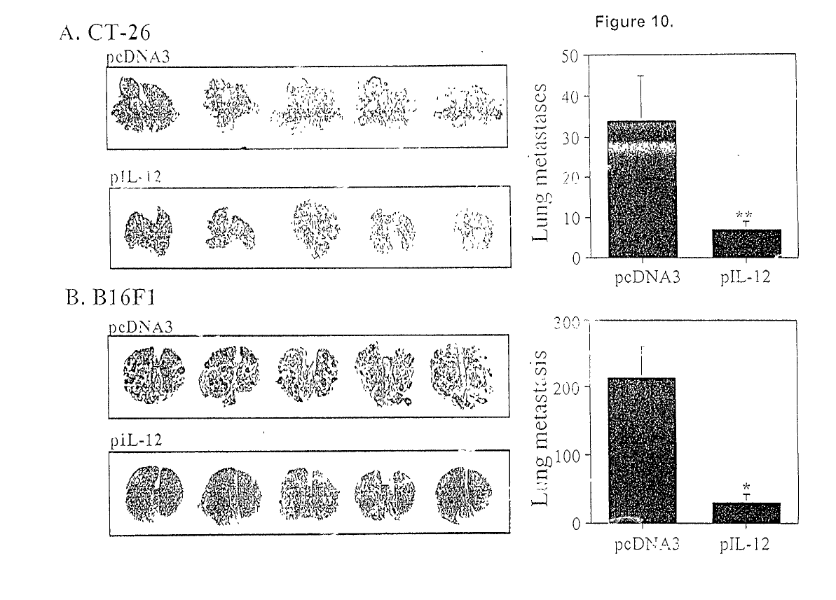

To evaluate the antimetastatic effect of pIL-12 electro gene therapy, pulmonary metastases were induced by intravenous injection of 1×10⁵ CT-26 cells into BALB/c mice or 2×10⁵ B16F1 cells into C57BL/6 mice. After 3 days, microscopically established metastases were present throughout the lung tissue. Grossly visible metastases were detectable on the surface of the lungs 21 days after tumor cell injection (experimental observation, data not shown). A single intramuscular electrotransfer of 100 μg of pIL-12 at day 3 markedly reduced the macroscopic metastatic lung foci in both the B16F1 and CT-26 tumor models (FIG. 10). In the B16F1 tumor model, mice that received pIL-12 by in vivo EP had a mean number of metastatic foci of 29±12 compared with 214±47 in the pcDNA3 control group (P<0.01) (FIG. 10A). In mice bearing CT-26 tumors, significant suppression of lung meatastasis was also achieved by EP-mediated IL-12 gene therapy (mean number of metastatic foci 7±2 versus 34±11 for the pcDNA3 control group, P<0.05) (FIG. 10B). In addition, the size of the existing tumor nodules in the pIL-12-treated group was much smaller than that of the pcDNA3 control group, indicating that the growth of metastatic tumors was suppressed by IL-12 electro gene treatment. These results demonstrate that intramuscular electrotransfer of pIL-12 was an effective therapy against both subcutaneous and metastatic tumors of different organ's origins.

Comparison of Different Cytokine Genes for their Ability to Inhibit 38C13 Tumor Growth by In Vivo EP

We next compared six different cytokine genes and one chemokine gene for their antitumor activity by in vivo EP. Mice were s.c. injected with 1×10³ 38C13 cells and treated 3 days later with 100 μg of plasmid encoding murine IL-12, IFN-γ, IL-18, IL-2, IL-4, TCA3, or GM-CSF followed by EP. Mice receiving 100 μg of pcDNA3 were included as controls. As summarized in Table I, in vivo EP delivery of pIL-12 led to tumor suppression (49.2±14.9 days versus 19.6±1.3 days of the pcDNA3 control group, P<0.01) and resulted in 60% (3 of 5 mice) long-term survivors. In vivo EP delivery of pIL-4 also led to tumor suppression to some extent (mean survival time, 26.0±6.6 days versus 19.6±1.3 of the pcDNA3 control group, P<0.05) but did not produce any long-term survivors. In vivo EP delivery of other cytokine genes, including IFN-γ, IL-18, IL-2, or GM-CSF, or chemokine TCA3 gene did not show inhibition of 38C13 tumor growth or prolongation of the survival time of mice bearing s.c. 38C13 tumor (Table I). This result demonstrates that in vivo EP provided a simple method for screening antitumor activities of potential therapeutic proteins.

Part II. Suppression of 38C13 Tumor Growth by i.m. Electrotransfer of Immunocytokines Genes

Immunocytokines are fusion proteins consisting of a tumor-specific monoclonal antibody and a cytokine molecule (18). It was demonstrated that immunocytokines could specifically target tumor cells and direct the attached cytokines to the tumor site. This specific targeting ability of immunocytokines should further enhance the therapeutic effect of cytokines. However, the process of production and purification of immunocytokines are tedious and expensive. We demonstrated here that i.m. electrotransfer of plasmids encoding tumor-specific immunocytokine genes provide a simple method to produce therapeutic levels of immunocytokines.

Construction of Plasmids Encoding Anti-idiotype-GM-CSF Immunocytokine

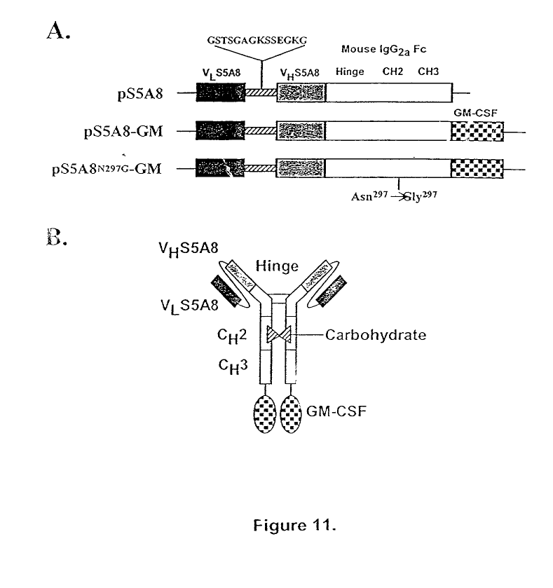

We used 38C13 B-cell lymphoma as a model system in this study. The idiotype (Id) of the surface immunoglobulin expressed by 38C13 tumor cells can serve as a unique tumor specific antigen (20). Several immunocytokines constructs were made (FIG. 11). All constructs contain the V_{L} and V_{H} domains of a 38C13 idiotypic protein-specific monoclonal antibody S5A8. In pS5A8 plasmid, the V_{L}-V_{H} sequence was ligated to the gene sequence encoding the hinge-C_{H}2-C_{H}3 of murine IgG2a. In pS5A8-GM plasmid, the murine GM-CSF sequence was ligated to the 3′-end of IgG C_{H}3 in pS5A8. The single-chain S5A8 V_{L}-V_{H} in these immunocytokines serves as a tumor-specific targeting domain that can deliver the GM-CSF molecule to the local tumor location. We also introduce a point mutation in pS5A8-GM to change Asn²⁹⁷ to Gly to eliminate the conserved IgG N-linked carbohydrates, which have been shown to be important for IgG's binding to the Fcγ receptor and complements. The resulted plasmid was designated as pS5A8^{N297G}-GM. We believe that an immunocytokine without non-specific binding to Fcγ receptor-bearing cells may possess improved tumor-targeting ability.

In vitro Expression of Immunocytokine Plasmids

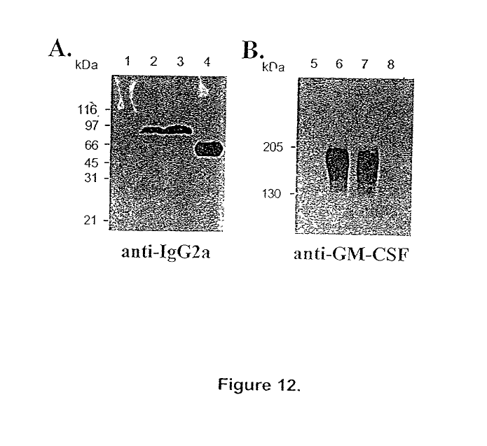

BALB/3T3 cells were transiently transfected with plasmids pS5A8, pS5A8-GM, or pS5A8^{N297G}-GM with the parental plasmid p3224-3 serving as a negative control. At one day after transfection, the protein products in the transfected cells were analyzed by immunoblotting techniques. In a reducing gel, plasmid pS5A8 expressed a protein protein product with an apparent molecular mass of 65 kDa (FIG. 12A, lane 4). Plasmids pS5A8-GM- or pS₅A₈^{N297G}-GM produced proteins with an apparent molecular mass of 80 kDa (FIG. 12A, lanes 2 and 3). The presence of GM-CSF molecule in the fusion proteins expressed by pS5A8-GM- or pS₅A8^{N297G}-GM was confirmed by their interaction with anti-murine GM-CSF antibody (FIG. 12B, lanes 6 and 7). In the nonreducing gel, these S5A8-GM-CSF fusion proteins migrated at an apparent molecular mass of 160 kDa, indicating that they are present in a dimeric form.

Id-binding and GM-CSF Biological Activities of S5A8-GM-CSF Immunocytokines

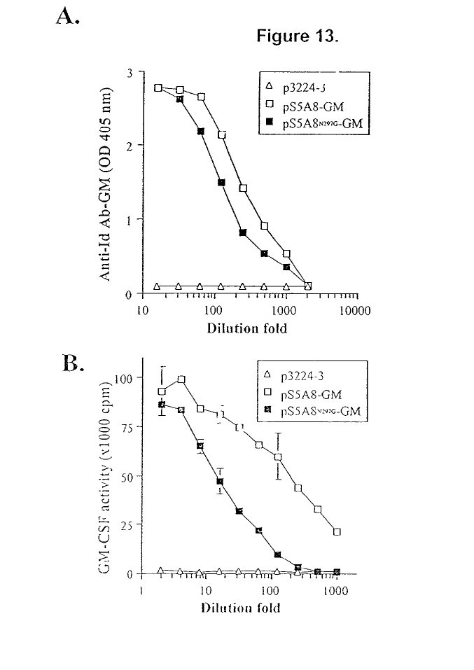

To confirm that the immunocytokines proteins retains the immunoractivity against the tumor Id protein, BALB/3T3 cells were transfected with plasmids pS5A8-GM- and pS5A8^{N297G}-GM and the supernatant were analyzed by an Id/anti-GM-CSF sandwich ELISA. The ELISA plates were coated with idiotypic proteins and the bound proteins were detected with anti-GM-CSF antibody. As shown in FIG. 13A, immunocytokines produced by plasmids pS5A8-GM- and pS5A8^{N297G}-GM clearly demonstrated the ability to bind to Id protein and contain GM-CSF molecule. The control plasmid did not produce proteins that are detectable in this assay.

Biological Activity of S5A8-GM-CSF Immunocytokines

To determine GM-CSF activity of the immunocytokines, supernatants from plasmids transfected BALB/3T3 cells were analyzed for their ability to support the proliferation of a murine GM-CSF-dependent cell line NFS-60. Supernatant from p3224-3-transfected cells was completely negative in this assay. In contrast, both S5A8-GM- and S5A8^{N297G}-GM immunocytokines clearly demonstrated the ability to stimulate the growth of NFS-60 cells in a dose-dependent manner. (FIG. 13B). These results demonstrate that the GM-CSF moiety of the fusion proteins produced by pS5A8-GM or pS5A8^{N297G}-GM plasmids was in a functional configuration.

In vivo Expression of S5A8-GM-CSF Immunocytokines Genes

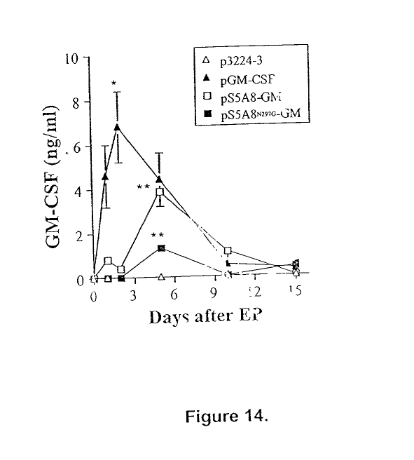

To evaluate these S5A8-GM-CSF plasmids as potential agents for cancer gene therapy, we tested their in vivo expression by intramuscular EP. C3H/HeN mice were injected in the TA muscles with 50 μg of pGM-CSF, pS5A8-GM or pS5A8^{N297G}-GM, and one group of mice was electrostimulated immediately after injection. Mice that received the control plasmid p3224-3 followed by electrostimulation served as negative controls. The time course of gene expression was determined by following muscle GM-CSF levels. As shown in FIG. 14, no GM-CSF levels in mice receiving the control plasmid p3224-3 were detectable within the sensitivity limit of the commercial ELISA assay. Mice in the pS5A8-GM- and pS5A8^{N297G}-GM gene-treated but unstimulated groups also did not produce detectable serum GM-CSF (data not shown). In contrast, both immunocytokines plasmids produced significant levels of GM-CSF proteins after EP stimulation. In the pS5A8-GM group, the muscle level of GM-CSF reached a peak level of 4 ng/ml at day 5. In the pS5A8^{N297G}-GM group, a low but significant level of immunocytokine expression (˜1.5 ng/ml on day 5) was present in the muscle. For comparison, pGM-CSF plasmid produced detectable GM-CSF from day 1 to day 10, with a peak level of 6.8 ng/ml on day 2. These results demonstrate that intramuscular EP of immunocytokines genes help produce proteins in vivo.

EP-mediated Transfer of S5A8-GM-CSF Immunocytokine Genes Inhibits 38C13 Tumor Growth

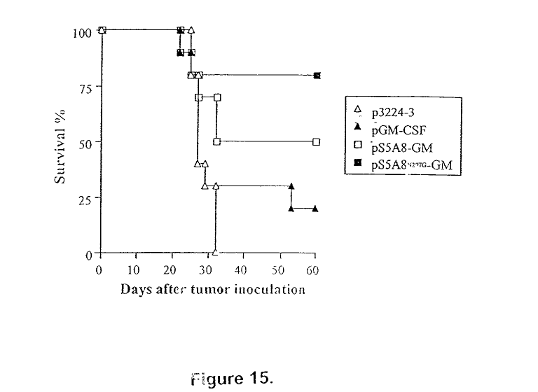

The antitumor effect of immunocytokine gene electrotransfer was next evaluated. Syngeneic C3H/HeN mice were inoculated s.c. with 1×10³ tumor cells at day 0. One day later, pGM-CSF, pS5A8-GM, or pS5A8^{N297G}-GM at doses of 100 μg was injected into the TA muscles followed immediately by in vivo EP. Mice treated with 100 μg of empty vector (p3224-3) alone were included as controls. The percentage of survivors is shown in FIG. 15 and Table II. Intramuscular EP of 100 μg of pS5A8-GM and pS5A8^{N297G}-GM resulted in 50% (5 of 10 mice) and 80% (8 of 10 mice) long-term survivors (>60 days), respectively, whereas all animals in the control p3224-3 group were dead within 32 days. Treatment with pGM-CSF by intramuscular EP produced a lower level of therapeutic effect, with 20% (2 of 10 mice) animals survived the tumor challenge. We also found that electroporation is required for the therapeutic effect of the S5A8-GM-CSF immunocytokines genes since a simple muscle injection of pS5A8-GM and pS5A8^{N297G}-GM did not show significant inhibition of tumor growth and produced no long-term survivors (not shown).

We then performed a more stringent experiment with more established tumor to assess the power of the S5A8-GM-CSF electro gene therapy. In mice bearing day 3 s.c. tumors, treatment with 100 μg of the pS5A8-GM or the pGM-CSF plasmids by in vivo EP did not show inhibition of 38C13 tumor growth (mean survival time 24±2 days and 23±1 days, respectively) as compared with mice receiving i.m. electrotransfer of 100 μg of the control plasmid p3224-3 (mean survival time 21±1 days) (Table II). Interestingly, i.m. electrotransfer of the plasmid encoding N²⁹⁷G aglycosylated immunocytokine showed significant suppression of tumor growth (mean survival time >34±4 days) and resulted in 10% (2 of 20 mice) of long term survivors. These results suggest that electrotransfer of the tumor-targeting immunocytokine gene has better antitumor effect than a similar treatment of cytokine genes. Moreover, reduction of non-specific binding of the immunocytokine can further enhance its therapeutic effect.

In summary, we show that intramuscular electrotransfer cytokine or immunocytokine genes has potent antitumor effects. This approach is simple, inexpensive and can be applied to quickly screen potential therapeutic genes. Application of in vivo EP to transfer cytokine/immunocytokine genes may represent a novel therapeutic strategy for cancer treatment.

Thus, while there have shown and described and pointed out fundamental novel features of the invention as applied to a preferred embodiment thereof, it will be understood that various omissions and substitutions and changes in the form and details of the methods illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

All cited references are herein incorporated in their entireties by reference.

### REFERENCES

1. Bergman, Y. and Haimovich, J. Characterization of a carcinogen-induced murine B lymphocyte cell line of C3H/eB origin. Eur. J. Immunol., 7: 413-417, 1977.

2. Brattain, M. G., Strobel Stevens, J., Fine, D., Webb, M., Sarrif, A. M., Brunda, M. J., Rosenbaum, D., Stern, L., Mathiesen, I., Mir, L. M., Bureau, M. F., Gehl, J., Rangara, R., Rouy, D., Caillaud, J. M., Delaere, P., Branellec, D., Schwartz, B., and Scherman, D. Establishment of mouse colonic carcinoma cell lines with different metastatic properties. Cancer Res, 40: 2142-2146, 1980.

3. Brunda, M. J., Rosenbaum, D., and Stern, L. Inhibition of experimentally-induced murine metastases by recombinant alpha interferon: correlation between the modulatory effect of interferon treatment on natural killer cell activity and inhibition of metastases. Int J Cancer, 34: 421-426, 1984.

4. Chow, Y. H., Huang, W. L., Chi, W. K., Chu, Y. D., and Tao, M. H. Improvement of hepatitis B virus DNA vaccines by plasmids coexpressing hepatitis B surface antigen and interleukin-2. J. Virol., 71: 169-178, 1997.

5. Chow, Y. H., Chiang, B. L., Lee, Y. L., Chi, W. K., Lin, W. C., Chen, Y. T., and Tao, M. H. Development of Th1 and Th2 populations and the nature of immune responses to hepatitis B virus DNA vaccines can be modulated by codelivery of various cytokine genes. J. Immunol., 160: 1320-1329, 1998.

6. Liu, S. J., Sher, Y. P., Ting, C. C., Liao, K. W., Yu, C. P., and Tao, M. H. Treatment of B-cell lymphoma with chimeric IgG and single-chain Fv antibody-interleukin-2 fusion proteins. Blood, 92: 2103-2112, 1998.

7. Holmes, K. L., Palaszynski, E., Fredrickson, T. N., Morse, H. C. r., and Ihle, J. N. Correlation of cell-surface phenotype with the establishment of interleukin 3-dependent cell lines from wild-mouse murine leukemia virus-induced neoplasms. Proc. Natl. Acad. Sci. U.S.A., 82: 6687-6691, 1985.

8. Hann, H. W., Stahlhut, M. W., Rubin, R., and Maddrey, W. C. Antitumor effect of deferoxamine on human hepatocellular carcinoma growing in athymic nude mice. Cancer, 70: 2051-2056, 1992.

9. Mathiesen, I., Tsantili, P., Tzartos, S. J., Mamalaki, A., Brunda, M. J., Rosenbaum, D., and Stern, L. Electropermeabilization of skeletal muscle enhances gene transfer in vivo. Gene Ther, 6: 508-514, 1999.

10. Mir, L. M., Bureau, M. F., Gehl, J., Rangara, R., Rouy, D., Caillaud, J. M., Delaere, P., Branellec, D., Schwartz, B., and Scherman, D. High-efficiency gene transfer into skeletal muscle mediated by electric pulses. Proc Natl Acad Sci USA, 96: 4262-4267, 1999.

11. Aihara, H. and Miyazaki, J. Gene transfer into muscle by electroporation in vivo. Nat. Biotechnol., 16: 867-870, 1998.

12. Gately, M. K., Renzetti, L. M., Magram, J., Stern, A. S., Adorini, L., Gubler, U., and Presky, D. H. The interleukin-12/interleukin-12-receptor system: role in normal and pathologic immune responses. Annu. Rev. Immunol., 16: 495-521, 1998.

13. Rakhmilevich, A. L., Turner, J., Ford, M. J., McCabe, D., Sun, W. H., Sondel, P. M., Grota, K., and Yang, N. S. Gene gun-mediated skin transfection with interleukin 12 gene results in regression of established primary and metastatic murine tumors. Proc. Natl. Acad. Sci. USA, 93: 6291-6296, 1996.

14. Orange, J. S., Salazar Mather, T. P., Opal, S. M., Spencer, R. L., Miller, A. H., McEwen, B. S., and Biron, C. A. Mechanism of interleukin 12-mediated toxicities during experimental viral infections: role of tumor necrosis factor and glucocorticoids. J. Exp. Med., 181: 901-914, 1995.

15. Rols, M. P., Delteil, C., Golzio, M., Dumond, P., Cros, S., and Teissie, J. In vivo electrically mediated protein and gene transfer in murine melanoma. Nat. Biotechnol., 16: 168-171, 1998.

16. Nishi, T., Yoshizato, K., Yamashiro, S., Takeshima, H., Sato, K., Hamada, K., Kitamura, I., Yoshimura, T., Saya, H., Kuratsu, J., and Ushio, Y. High-efficiency in vivo gene transfer using intraarterial plasmid DNA injection following in vivo electroporation. Cancer Res., 56: 1050-1055, 1996.

17. Goto, T., Nishi, T., Tamura, T., Dev, S. B., Takeshima, H., Kochi, M., Yoshizato, K., Kuratsu, J., Sakata, T., Hofinann, G. A., and Ushio, Y. Highly efficient electro-gene therapy of solid tumor by using an expression plasmid for the herpes simplex virus thymidine kinase gene. Proc. Natl. Acad. Sci. USA, 97: 354-359, 2000.

18. Lode, H. N. and Reisfeld, R. A. Targeted cytokines for cancer immunotherapy. Immunol. Res., 21: 279-288, 2000.

19. Peng, L. S., Penichet, M. L., and Morrison, S. L. A single-chain IL-12 IgG3 antibody fusion protein retains antibody specificity and IL-12 bioactivity and demonstrates antitumor activity. J. Inmunol., 163: 250-258, 1999.

20. Perek, Y., Hurwitz, E., Burowski, D., and Haimovich, J. Immunotherapy of a murine B cell tumor with antibodies and F(ab′)2 fragements against idiotypic determinants of its cell surface IgM. J. Immunol., 131: 1600-1603, 1983.

TABLE I
The survival of mice bearing s.c. 38C13 tumor after i.m. electrotransfer
of cytokine gene.

| | | Mice with complete | |
|---|---|---|---|
| Treatment | Mean survival time (days) | regression*/total | Percentage |
| pTCAE | 19.6 ± 1.3 | 0/5 | 0% |
| pIL-12 | 49.2 ± 14.9** | 3/5 | 60% |
| pIFN-γ | 22.0 ± 4.3 | 0/5 | 0% |
| pIL-18 | 24.0 ± 7.0 | 0/5 | 0% |
| pIL-2 | 18.2 ± 1.1 | 0/5 | 0% |
| pIL-4 | 26.0 ± 6.6*** | 0/5 | 0% |
| pTCA3 | 22.8 ± 3.6 | 0/5 | 0% |
| pGM-CSF | 24.4 ± 13.2 | 0/5 | 0% |

Treatment protocol was same as that of FIG. 7.
*The number of mice in which complete regression of s.c. 38C13 tumor was observed.
**p < 0.01 and
***p < 0.05 vs. pTCAE.

We claim:

1. A method of treating cancer in a mammal comprising delivering by electroporation an immunocytokine gene in an expression plasmid into cells of the mammal.

2. The method of claim 1 wherein the cells of the mammal are muscle or cancer cells.

3. The method of claim 1 wherein the immunocytokine gene codes for cytokine selected from the group consisting of interleukin-1, interleukin-2, interleukin-3, interleukin-4, interleukin-5, interleukin-6, interleukin-12, interleukin-18, gamma-interferon and GM-CSF.

4. The method of claim 3 wherein the immunocytokine gene codes for GM-CSF.

5. The method of claim 1 wherein the immunocytokine gene codes for a monoclonal antibody specific to an antigen of the cancer cells.

6. The method of claim 5 wherein the monoclonal antibody is an IgG.

7. The method of claim 6 wherein the IgG monoclonal antibody is S5A8.

8. The method of claim 1 wherein the immunocytokine gene has a point mutation that causes the removal of CH2-linked carbohydrate from the immunocytokine.

9. The method of claim 1 wherein the expression plasmid has a CMV promoter.

10. A method for stimulating an immune response against a tumor in a patient, comprising delivering by electroporation into muscle cells of the patient a plasmid containing a gene coding for S5A8^{N297G}-GM-CSF to produce transformed muscle cells.

11. A method of treating cancer in a mammal comprising delivering by electroporation a cytokine gene in an expression plasmid into muscle cells of the mammal.

12. The method of claim 11 wherein the delivery is at a site near an active cancer site.
